(19) United States
(12) Patent Application Publication
(10) Pub. No.: US 2026/0176766 A1
(43) Pub. Date: Jun. 25, 2026

(54) METHOD OF MANUFACTURING NEBULISER APERTURE PLATES

(71) Applicant: STAMFORD DEVICES LIMITED, GALWAY (IE)

(72) Inventor: Cian MCKEOWN, Limerick (IE)

(73) Assignee: STAMFORD DEVICES LIMITED, GALWAY (IE)

(21) Appl. No.: 19/126,708

(22) PCT Filed: Oct. 18, 2023
(86) PCT No.: PCT/EP2023/079042

§ 371 Date: May 2, 2025

(30) Foreign Application Priority Data
Nov. 3, 2022 (EP) 22205355.5

(51) Int. Cl. C23C 18/16; A61M 11/00; B05B 17/06; C23C 18/30; C23C 18/50

(52) U.S. Cl. CPC C23C 18/1657; A61M 11/005; B05B 17/06; C23C 18/1605; C23C 18/1641; C23C 18/1648; C23C 18/30; C23C 18/50

(57) ABSTRACT

A method of producing an aperture plate for a nebuliser is described. A substrate (2) is patterned with a resist (5, 6) and a metal (10) is applied by electroless deposition so that the resist forms a desired aperture pattern. The substrate (2) has an array of domes (2(b)) to provide aperture plates (16) with domes (101) and flanges (102) without need for application of mechanical force pressing after deposition. The substrate is shaped for simultaneous manufacture of a plurality of aperture plates in which the resist pattern provides a deposition region for each aperture plate. By using electroless plating the aperture plate accuracy is excellent, especially for defining apertures having a diameter in the rang of only less than 10 μm, and having improved corrosion and fracture resistance for high frequency nebulizer operation.

### INTRODUCTION

The present invention relates to a method of manufacturing nebuliser aperture plates.

U.S. Pat. No. 6,235,177 (Aerogen) describes a manufacturing method which includes electroplating in which a wafer material is built onto a mandrel by a process of electrodeposition. Another example of electroforming is described in U.S. Pat. No. 5,685,491 (AMTX, Inc.), which describes in detail electroforming with an anode and a cathode, the product being a spray director.

WO2013/186031 and EP2947181 (Stamford Devices Limited) describe electrodeposition approaches with photo defined patterns to provide aerosol forming apertures and a reservoir layer with larger supply holes.

The invention is directed towards providing improved uniformity of aperture plates with three-dimensional structures which are optimised for aerosol-producing operation when vibrated at frequencies in the region of 128 kHz. For example, a three-dimensional shape may be a dome with the aerosol-producing apertures and a rim in the form of a flange for attachment to a washer-shaped support. The depth of the dome portion and its radius of curvature may be important to achieve the desired aerosol flow rate and droplet size for access of the aerosol deep into the lungs of a patient.

### SUMMARY OF THE INVENTION

We describe a method of producing an aperture plate for a nebuliser, the method comprising steps of:
providing a substrate,
patterning the substrate with a resist having positive features corresponding to aerosol-forming apertures,
in a deposition bath performing electroless deposition of a metal onto the substrate where the substrate is not covered by the resist,
removing the resist to reveal a metal layer with apertures where the resist was previously, and
removing the substrate to provide an aperture plate.

In this way the remaining metal after removal of the substrate is itself the aperture plate, which may be fully formed without need for any further operations such as stamping. The electroless deposition approach provides the full three-dimensional shape and configuration and accuracy of aerosol-forming hole manufacture.

In some embodiments, the substrate is shaped with a three-dimensional shape to provide an aperture plate without need for application of mechanical force after deposition. In some embodiments, the substrate is dome shaped, and the resist is applied so the deposited metal forms each aperture plate with a dome and a surrounding flange. In some embodiments, the substrate is shaped for simultaneous manufacture of a plurality of aperture plates in which the resist pattern provides a deposition region for each aperture plate.

In some embodiments, the method comprises a subsequent series of steps of patterning a subsequent resist and depositing by electroless deposition a second layer of metal to provide liquid supply cavities, at least some of which overlie a plurality of aerosol-forming apertures provided by a first series of resist patterning and electroless plating steps. In some embodiments, the substate is of a polycarbonate material. In some embodiments, the substrate is dissolved for removal, for example by being submerged in organic solvent dichloromethane (CH₂Cl₂).

In some embodiments, a substrate surface is sensitized and then is activated with nucleation sites. In some embodiments, the sensitizing is performed by immersion in a bath of a solution such a tin chloride. In some embodiments, the nucleation activation is performed by immersion in a bath of a solution such as palladium chloride. In some embodiments, the metal comprises Ni. In some embodiments, the metal comprises Ni—Pd.

In some embodiments, for sensitizing, the substrate is submerged in a tin (Sn) sensitisation bath containing 0.013 M SnCl₂ powder dissolved in 0.24 HCl for a period in the range of 5 to 15, for example 10 minutes. In some embodiments, the substrate is rinsed after being sensitized and before being transferred to an activation bath containing 0.0014 M PdCl₂ dissolved in D.I. water.

In some embodiments, the activation bath temperature is maintained at a temperature in the range of 60° C. to 70° C., preferably about 65° C.

In some embodiments, the metal deposition is performed using a bath comprising a source of metal ions, a reducing agent, a complexing agent, and pH buffers. In some embodiments, the metal ions include NiSO₄, and/or NiCl₂, and/or CoSO₄, and/or Fe SO₄.

In some embodiments, the reducing agent comprises dimethylamineborane (DMAB). In some embodiments, the reducing agent comprises sodium hypophosphite (NaPO₂H₂). In some embodiments, the complexing agent comprises lactic acid. In some embodiments, the complexing agent comprises ammonium citrate. In some embodiments, the pH buffers comprise NaOH, and/or HCl and/or boric acid, and/or triethanolamine. In some embodiments, the deposition bath pH is adjusted to about pH9. In some embodiments, the deposition bath deposition temperature is maintained at a temperature in the range of 35° C. to 45° C., preferably 40° C. In some embodiments, the substrate is ion-track etched polycarbonate (PC) material.

We also describe a nebulizer aperture plate comprising aerosol-forming apertures in a dome-shaped portion of the plate and a surrounding flange for attachment to an annular support, the aperture plate having the characteristics of being integrally formed with the dome and flange structure by electroless deposition.

Preferably, the aerosol-forming apertures have a diameter in the range of 2 μm to 6 μm.

In some examples, the plate includes Ni.

We also describe a nebulizer comprising:
a housing comprising a liquid supply reservoir,
an aerosol outlet formed by the housing,
an aerosol generator mounted in the housing and comprising:
   a vibratable aperture plate of any example described herein,
   an annular support supporting the aperture plate,
   a vibration generator attached to the annular support,
   a power conductor for transferring power to the vibration generator,
   a downstream resilient seal mounted between the housing and the annular support on a side of the aperture plate opposed to the liquid supply reservoir, and
   an upstream resilient seal mounted between the annular support and the housing reservoir and having an opening with an exposed surface forming part of a throat over the aperture plate.

### DETAILED DESCRIPTION OF THE INVENTION

The invention will be more clearly understood from the following description of some embodiments thereof, given by way of example only with reference to the accompanying drawings in which:

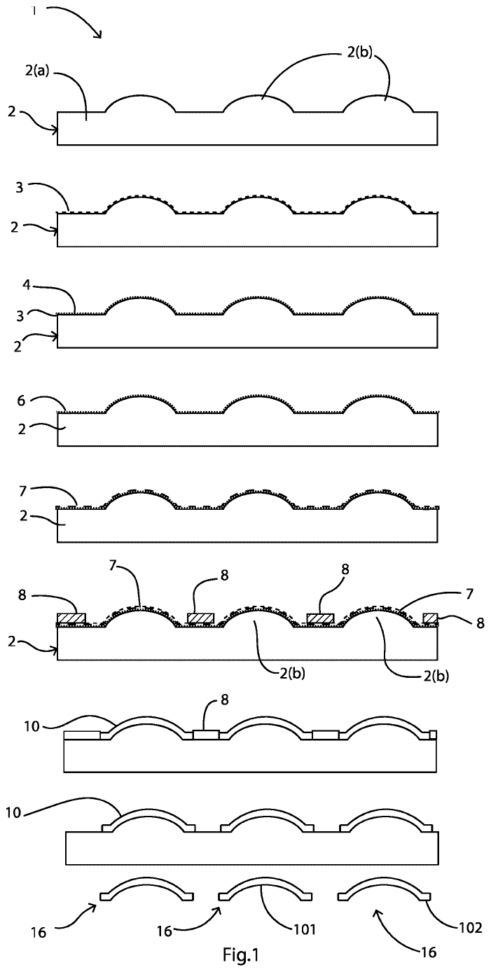

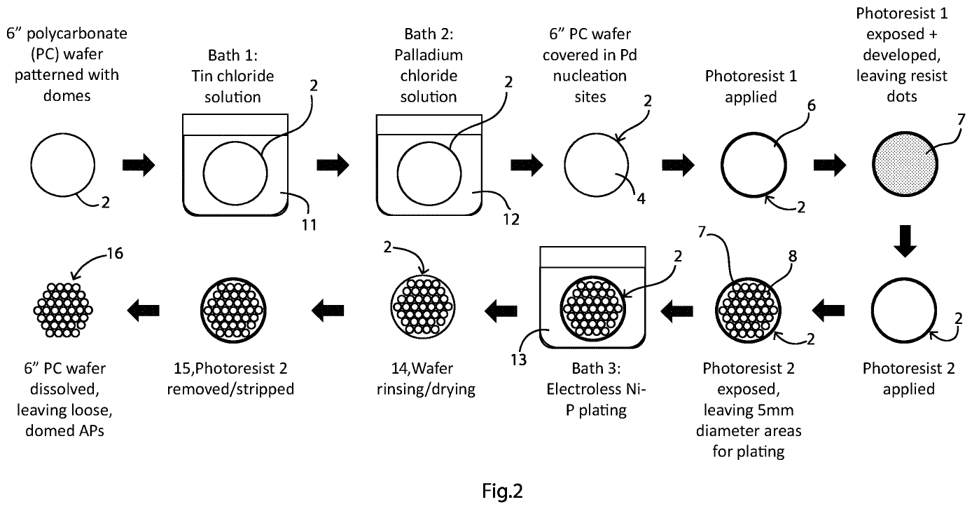

FIG. 1 is a process flow diagram of a method of manufacturing an aperture plate, and FIG. 2 shows the process flow with plan views of a wafer during the stages of manufacture,

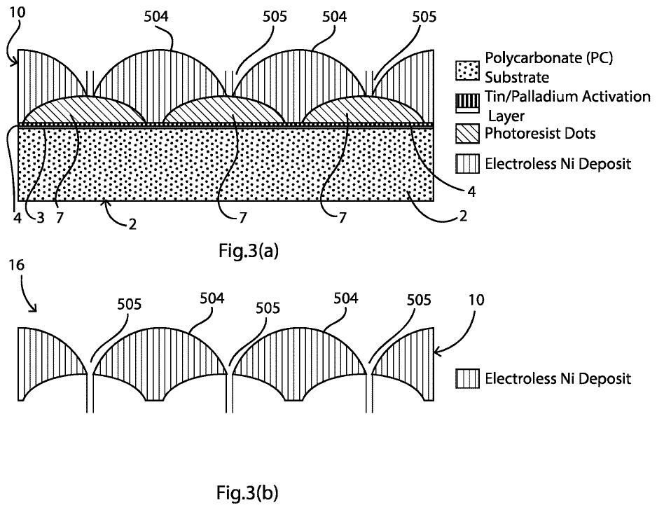

FIG. 3(a) is a cross-sectional diagram showing the aperture plate during manufacture at the level of individual apertures in detail for one example; and FIG. 3(b) is a cross-sectional view of the resulting aperture plate at this location,

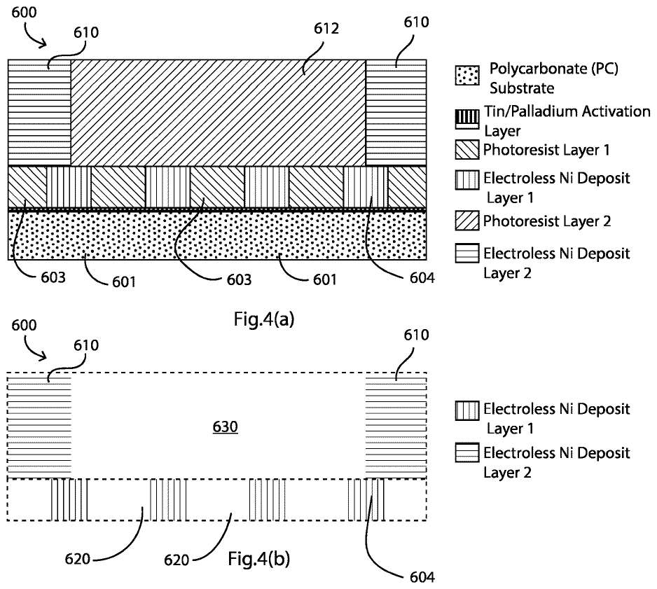

FIG. 4(a) is a cross-sectional diagram showing the aperture plate at the level of individual apertures and liquid supply cavities during manufacture in detail for another example; and FIG. 4(b) is a cross-sectional view of the resulting aperture plate at this location; and

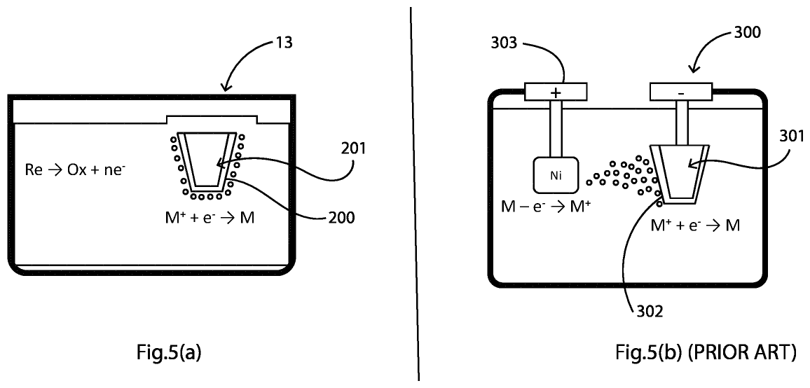

FIG. 5(a) and 5(b) are diagrams illustrating the differences between plating in accordance with the invention and prior art electroplating, respectively.

### DETAILED DESCRIPTION OF THE EMBODIMENTS

We describe a method of manufacturing aperture plates for nebulizers using a chemical technique known as electroless deposition or electroless plating. This method produces effective aperture plates with the required characteristics such as thickness, aerosol-forming aperture dimensions, and overall aperture plate shape. Using this method, there is no requirement for an external power supply to initiate the reduction of metal ions in a plating bath, and individual aperture plates can be grown onto a pre-patterned, domed, or flat substrate. This avoids need for any forming operation after plating to provide a three-dimensional shape such as a domed shape. The invention provides a process that produces identical aperture plates (fixed thickness, composition, outside diameter, dome height, dome diameter), ready to be inserted into nebuliser cores directly from the plating bath. There is no need for steps such as stamping or cutting after deposition.

The method of the invention involves a two-step photolithography process to create two distinct photoresist patterns. The first is a pattern marking out 5 mm diameter regions where the electroless deposition will take place, one region per aperture plate. The second is a pattern of resist dots and pillars within these regions, to create the aerosol-forming apertures for the aperture plate. This allows a number aperture plates to be formed simultaneously on the same polymer substrate, the second patterning step defining the aperture plate regions.

### FIG. 1

Referring to FIG. 1, a polycarbonate substrate 2 has a planar portion 2(a) and domes 2(b), and has a minimum thickness of 0.5 mm, and a 150 mm diameter, and is patterned with domes of fixed dimensions. The substate domes 2(b) in one example have a diameter of 4.4 mm and a height of 0.35 mm. The pattern can be formed by laser etching, ion polishing, or selective chemical etching. In one example there are approximately 900 domes, one per aperture plate to be manufactured.

A Sn²⁺ sensitisation layer 3 is applied to the substrate, and a Pd activation layer 4 is applied on top. From this, a first photoresist layer is applied with photoresist patterned pillars 6.

There is then re-flow so that the pillars 6 form domed dots 7. These are shown in best detail in FIG. 3(a).

A second photoresist layer 8 is then applied to define the substrate regions, each of which is for an individual aperture plate. The second photoresist layer 8 forms spaces around the deposition regions so that there is deposition only within the regions (on the domes 2(b)).

There is then electroless deposition to provide a metal layer 10 for each aperture plate. Electroless deposition does not take place between the regions.

All of the photoresist is then stripped, and the polycarbonate substrate 2 is dissolved using Dichloromethane to leave only the end-product aperture plates 16. An aperture plate 16 is illustrated at the level of individual apertures in FIG. 3(b). The metal layer 10 comprises raised humps 504 of Ni deposits defining an array of apertures 505. The dots 504 are shaped to provide a funnel shape leading into the apertures 505 on the liquid inlet side. In terms of the overall physical configuration, the end result is akin to that achieved in the known electroforming approach. However, the manufacture as described with electroless deposition allows the full aperture plate dome shape to be achieved in the plating process without need for subsequent stamping operations. This three-dimensional shape includes, in the aperture plate 16 shows in FIG. 1, a dome 101 with the active aerosol-forming apertures and a flange 102 for attachment to a washer-shaped support Also, the microstructure is different and beneficial to corrosion and fatigue resistance because of its amorphous or semi-crystalline structure. The semi crystalline structure is due to the minor amounts of phosphorus or boron in the atomic lattice, which disrupt the growth of large grains of pure Ni. Both corrosion and fracture initiation occur at grain boundaries, which are not readily available in amorphous or semi-crystalline materials.

### FIG. 2

Referring to FIG. 2, the process is shown in this case with plan view diagrams. The 150 mm diameter polycarbonate substrate 2 is shown initially.

The substrate 2 is then submerged in a bath of tin chloride solution 11 at 40° C. for 10 minutes containing 0.013 M SnCl2 and 0.24 M HCl. This provides the sensitisation layer 3.

The substrate 2 is then rinsed in D.I. water, before immersion in a palladium chloride activation bath 12 at 65° C. for 10 minutes containing: 0.0014 M PdCl2 in D.I. water. The activated substrate 2 is then removed from the PdCl2 solution and dried in an oven. The substrate 2 is covered in palladium Pd nucleation sites 4 as an activation layer.

The first photoresist layer is then applied, and is UV cured through a mask to provide a pattern of cylindrical pillars 6 with a diameter of 120 μm and a height of 30 μm. The substrate 2 with the photoresist pillars 6 is then placed in a reflow oven at 350° C. to transform the pillars 6 of photoresist into hemispherical photoresist ‘dots’ 7. The dots 7 are shown in more detail in FIG. 3(a). This photoresist layer is then allowed to develop and is exposed, leaving the resist dots 7.

A second layer of photoresist 8 is then applied uniformly across the entire surface of the substrate, adhering to the shape of the domed regions. This is applied using a controllable spin coating process for non-flat substrates. A second photolithography mask is placed above the layer of photoresist 8 and exposed to UV light to mark out the circular regions around the domes on the substrate surface. This photoresist layer initially covers the entire surface, including the domed regions. Only after the mask is used during the UV exposure do the regions get marked out from that photoresist.

The substrate 2 is then placed in an electroless plating (third) bath 13, in which there is electroless Ni—P plating of the layer 10 on the domed substrate regions 2(b), before being rinsed and dried in step 14.

The electroless plating bath can include any combination of the below, held at a suitable temperature and pH to ensure a controllable deposition rate:
A source of metal ions (e.g. NiSO₄)
A reducing agent (e.g. dimethylamineborane (DMAB) if plating Ni—B, sodium hypophosphite (NaPO₂H₂) if plating Ni—P)
A complexing agent (e.g. lactic acid, ammonium citrate)
pH buffers for providing pH stability

In step 15 the substrate 2 is then removed from the deposition bath, rinsed in D.I. and dried, and second photoresist 8 is removed and stripped from the substrate.

The substrate 2 is then dissolved to release the domed aperture plates 16. This involves submerging the substrate in a strong solvent (e.g. dichloromethane) to dissolve the polycarbonate. This results in an array of loose and domed aperture plates 16.

With choice of photoresist patterns different configurations of aperture plate may be manufactured. For example, as show in in FIG. 4(a) a polycarbonate substrate 601 is coated as described above with a sensitization and activation layers before application of a first resist pattern of which defines columns 603 of resist where the aerosol-forming apertures are to be. There is Ni electroless plating 604 around these columns, thereby providing a first layer of metal with aerosol-forming apertures 620

Then, a second photoresist pattern of larger liquid-supply columns 612 is applied, each overlying a number of the apertures 620. After electroless deposition of a Ni layer 610 there are liquid supply cavities 630, together providing a reservoir layer over the aerosol-forming apertures 620.

In this example the parameters to of the end product aperture plate 600 are:
Number of apertures 620: 600.
Number of liquid supply cavities: 630, 200.
Diameter of aerosol-forming apertures 620: 2-3 μm.
Diameter of liquid supply cavities 630: 30 μm.
Thickness of the plate 600: 60 μm.

### EXAMPLES

The following are examples of performance of stages of the method described above.

### Tin Sensitisation and Pd Activation

### Example 1

Acetate sheets with dimensions 40 mm (L)×40 (W) mm×0.2 mm (H) were first submerged in a tin (Sn) sensitisation bath (40 ml in volume) containing 0.013 M SnCl2 powder dissolved in 0.24 HCl for 10 minutes. The bath was kept at 40° C. The acetate sheets were then rinsed and transferred to a palladium (Pd) activation bath (40 ml in volume) containing 0.0014 M PdCl2 dissolved in D.I. water. The bath was kept at 65° C. and the acetate sheets were submerged for 10 minutes. The sheets were then rinsed and transferred to an oven at 100° C. until dry. The sensitisation/activation process transformed the clear acetate to having a dark brown appearance, indicating the presence of Pd nuclei on the surface of the polymer sheet. The mass of the sheets was measured before and after sensitisation/activation and the increase in mass also indicated the presence of Pd nuclei on the surface. The acetate sheets were subsequently used as substrates in electroless deposition bath.

### Example 2

Ion-track etched polycarbonate (PC) membrane disks (30 mm diameter, 0.2 mm thickness) were first submerged in a tin (Sn) sensitisation bath (40 ml in volume) containing 0.013 M SnCl2 powder dissolved in 0.24 HCl for 10 minutes. The bath was kept at 40° C. The polycarbonate membranes were then rinsed and transferred to a palladium (Pd) activation bath (40 ml in volume) containing 0.0014 M PdCl2 dissolved in D.I. water. The bath was kept at 65° C. and the polycarbonate membranes were submerged for 10 minutes. The polycarbonate was then rinsed and transferred to an oven at 100° C. until dry. The sensitisation/activation process transformed the white polycarbonate to having a brown appearance, indicating the presence of Pd nuclei on the surface of the polymeric membrane. The mass of the polycarbonate was measured before and after sensitisation/activation and the increase in mass also indicated the presence of Pd nuclei on the surface. The polycarbonate templates were subsequently used as substrates in electroless deposition bath.

### Electroless Plating of Metal Films on Polymer Substrate

### Example 3

The activated acetate sheets from Example 1 were used as substrates for the electroless deposition of Ni—B metal sheets for use as aperture plates. The deposition bath comprised of 40 ml of lactic acid (CH₃CHCOOH), 0.052M ammonium citrate (C₆H₁₇N₃O₇), 0.07 M dimethylamine borane (DMAB), 0.1 M nickel sulphate (NiSO₄). The bath pH was adjusted to pH 9 using 1M NaOH. Deposition was carried out at 40° C. and the thickness of the Ni—B metal sheet was determined by the time in the plating bath. The time to achieve a thickness of 10 μm was about 1 hour, providing a uniform layer. If the substrate were patterned with cured resist the deposition parameters would be similar.

### Example 4

Same process as in Example 3 but 0.1 M cobalt sulphate (CoSO₄) replaced the 0.1 M nickel sulphate (NiSO₄) which produced an electrolessly deposited film of Co—B on a polymeric acetate substrate.

### Example 5

Same process as in Example 3 but 0.1 M iron sulphate (FeSO₄) replaced the 0.1 M nickel sulphate (NiSO₄) which produced an electrolessly deposited film of Fe-B on a polymeric acetate substrate.

### Electroless Plating of 3-D Metallic Structures in Dissolvable Polymer Templates

### Example 6

The activated polycarbonate templates from Example 2 were used as substrates for the electroless deposition of high-surface area metallic nanostructures with apertures on the submicron scale. The deposition bath described in Example 3 was used to create a network of metallic nanotubes within the pores of the ion-track etched polycarbonate (PC) template, which were joined by thin metal films at each end. Once deposition was complete, the sample was submerged in the organic solvent dichloromethane (CH₂Cl₂) and dissolved, leaving a free-standing network of metallic nanotubes as the end product. The pores of the substrate had an average diameter in the range of 0.1 μm to 1 μm, and this deposition is akin to deposition on a pattern of cured resist.

### Example 7

Same process as Example 6 but a different electroless bath chemistry was used to achieve high-surface area nanostructures of iron (Fe). The deposition bath comprised of 0.025-0.5 M iron sulphate (FeSO₄·7H₂O), 0.125-0.25 M sodium citrate (Na₃C₆H₅O₇), and 0.025-0.1 M sodium borohydride (NaBH₄) at a pH between 9.7 and 10.7 at a temperature of 40° C. Once deposition was complete, the sample was submerged in the organic solvent dichloromethane (CH₂Cl₂) and dissolved, leaving a free-standing network of metallic nanotubes as the end product.

### Advantages

To illustrate the benefits of the invention FIG. 5(a) shows the plating bath 13, with the electroless plating providing a uniform coating over a substrate 201. However, by contrast, in an electroplating bath 300 of the prior art there is a greater risk of a larger coating thickness 302 on the side of a substrate 301 closest to the positive electrode 303. The negative effect of electrode placement on the resultant electrodeposit may in some circumstances inhibit the uniform growth of metal films on irregularly shaped substrates using electrodeposition.

The electroless deposition (also known as electroless or autocatalytic plating) creates aperture plates via the autocatalytic chemical reduction of metal ions in a liquid bath, with no need for an external power supply. This has the major benefit of avoiding physical stamping to achieve a three dimensional shaped aperture plate, for example with a dome and a flange. It is a versatile method that can be used to grow a range of metals and alloys, such as electroless nickel (Ni). In some embodiments, due to the aqueous reducing agent in the bath, the resultant material contains small amounts of phosphorus or boron (2-15%) referred to as Ni—P or Ni—B. The invention allows one to tailor the level of phosphorus or boron in the alloy to give beneficial material properties including the properties of density, hardness, and resistivity/conductivity. Thus, the electroless bath chemistry can determine the material properties of the resultant aperture plates, rather than post-processing techniques such as annealing being required. Electroless deposits are amorphous (i.e. no defined crystal structure) which give them improved fracture resistance and corrosion resistance due to the lack of defined grain boundaries.

Upon manufacture each aperture plate is ready to be inserted into a nebulizer core such as described in our published applications WO2012/046220 or WO2021/191160 (Stamford Devices Ltd), the contents of which are incorporated herein by reference. The aperture plate may be inserted in a nebulizer having:
a housing and a liquid supply reservoir formed by the housing,
an aerosol outlet formed by the housing,
an aerosol generator mounted in the housing and comprising:
   a vibratable aperture plate having the characteristics of being formed by electroless plating in one of the examples described herein,
   an annular support supporting the aperture plate, typically in the form of a washer, possible of steel alloy material,
   a vibration generator attached to the annular support, for example an annular piezoelectric vibration generator secured to the annular support,
   a power conductor for transferring power to the vibration generator,
   a downstream resilient seal mounted between the housing and the annular support on a side of the aperture plate opposed to the liquid supply reservoir, and
   an upstream resilient seal mounted between the annular support and the housing reservoir and having an opening with an exposed surface forming part of a throat over the aperture plate.

The controller of such a nebulizer is typically arranged to drive the piezoelectric vibration generator at a frequency such as 128 kHz, and the environment is very moist with a liquid in the reservoir having a composition of the required medicine. An aperture plate manufactured by electroless plating as described is particularly suited to resisting corrosion and cracking in this environment. The aperture plate preferably has apertures with a diameter in the range of 2 μm and 6 μm, and electroless plating as described is particularly suited to manufacturing with accuracy with such small dimensions. The following summarises some of the major benefits of the invention:
High degree of control of material properties
Uniform deposition resulting in identical aperture plates and minimal yield loss.
Reduced device performance variation by minimising influence of multiple processes.
Versatile aperture geometry, for example, to enable nebulisation of different drugs.
Highly corrosion resistant compared with electroformed metal.
Increased fracture resistance compared with electroformed metal.

The components and method steps described above may be used in any combination which is effective as would be understood by a person of ordinary skill in the field. For example, the same masking and plating steps may be performed with a different substrate and/or different substrate removal method such as peeling off.

1. A method of producing an aperture plate for a nebuliser, the method comprising steps of:
providing a substrate, sensitizing a substrate surface and then activating said surface with nucleation sites,
patterning the substrate with a resist having positive features corresponding to aerosol-forming apertures,
in a deposition bath performing electroless deposition of a metal onto the substrate where the substrate is not covered by the resist,
removing the resist to reveal a metal layer with apertures where the resist was previously, and
removing the substrate to provide an aperture plate,
wherein the substrate is shaped with a three-dimensional shape to provide an aperture plate without need for application of mechanical force after deposition.

2. A method as claimed in claim 1, wherein the substrate is dome shaped, and the resist is applied such that the deposited metal forms each aperture plate (100) with a dome and a surrounding flange.

3. A method as claimed in claim 1, wherein the substrate is shaped for simultaneous manufacture of a plurality of aperture plates in which the resist pattern provides a deposition region for each aperture plate.

4. A method as claimed in claim 1, wherein the method comprises a subsequent series of steps of patterning a subsequent resist and depositing by electroless deposition a second layer of metal to provide liquid supply cavities, at least some of which overlie a plurality of aerosol-forming apertures provided by a first series of resist patterning and electroless plating steps.

5. A method as claimed in claim 1, wherein the substate is of a polycarbonate material.

6. A method as claimed in claim 1, wherein the substrate is dissolved for removal, for example by being submerged in organic solvent dichloromethane (CH₂Cl₂).

7. A method as claimed in claim 1, wherein, the substrate comprises an ion-track etched polycarbonate (PC) material.

8. A method as claimed in claim 1, wherein the sensitizing is performed by immersion in a bath of a solution, and optionally said solution comprises tin chloride.

9. A method as claimed in claim 1, wherein the nucleation activation is performed by immersion in a bath of a solution such as palladium chloride.

10. A method as claimed in claim 1, wherein the metal comprises Ni.

11. A method as claimed in claim 10, wherein the metal comprises Ni—Pd.

12. A method as claimed in claim 1 wherein, for sensitizing, the substrate is submerged in a tin (Sn) sensitisation bath containing 0.013 M SnCl₂ powder dissolved in 0.24 HCl for a period in the range of 5 to 15 minutes.

13. A method as claimed in claim 1, wherein the substrate is rinsed after being sensitized and before being transferred to an activation bath containing 0.0014 M PdCl₂ dissolved in D.I. water.

14. A method as claimed in claim 13, wherein the activation bath temperature is maintained at a temperature in the range of 60° C. to 70° C.

15. A method as claimed in claim 1, wherein the metal deposition is performed using a bath comprising a source of metal ions, a reducing agent, a complexing agent, and a pH buffer.

16. A method as claimed in claim 15, wherein the metal ions include NiSO₄, and/or NiCl₂, and/or CoSO₄, and/or Fe SO₄.

17. (canceled)

18. (canceled)

19. (canceled)

20. (canceled)

21. (canceled)

22. (canceled)

23. A nebulizer aperture plate comprising aerosol-forming apertures in a dome-shaped portion of the plate and a surrounding flange for attachment to an annular support, the aperture plate having the characteristics of being integrally formed with the dome and flange structure by electroless deposition.

24. An aperture plate as claimed in claim 23, wherein the aerosol-forming apertures have a diameter in the range of 2 μm to 6 μm.

25. An aperture plate as claimed in claim 24, wherein the plate includes Ni.

26. A nebulizer comprising:
a housing comprising a liquid supply reservoir,
an aerosol outlet formed by the housing,
an aerosol generator mounted in the housing and comprising:
a vibratable aperture plate of any of claim 25,
an annular support supporting the aperture plate,
a vibration generator attached to the annular support,
a power conductor for transferring power to the vibration generator,
a downstream resilient seal mounted between the housing and the annular support on a side of the aperture plate opposed to the liquid supply reservoir, and
an upstream resilient seal mounted between the annular support and the housing reservoir, and having an opening with an exposed surface forming part of a throat over the aperture plate.
